(19) United States
(12) Patent Application Publication
(10) Pub. No.: US 2003/0064123 A1
(43) Pub. Date: Apr. 3, 2003

(54) DHEA supplement for increasing women's libido

(76) Inventor: Ronald J. Thompson, Fort Thomas, KY (US)

Correspondence Address: Donald N. Halgren, 35 Central Street, Manchester, MA, 01944, US

(21) Appl. No.: 10/159,894

(22) Filed: May 30, 2002

Related U.S. Application Data
(63) Continuation-in-part of application No. 09/968,055, filed on Oct. 1, 2001.

(51) Int. Cl. A61K 35/78; A61K 31/4745; A61K 31/198; A61K 31/202

(52) U.S. Cl. 424/769; 514/283; 514/560; 514/565

(57) ABSTRACT

A method to increase both the libido and sexual responsiveness in a mammalian female. The method comprises the steps of administering a compound having a combination of DHEA, Yohimbe and L-arginine to that female. When a woman's decreased or absent libido, currently referred to in the medical literature as hypoactive sexual desire, is believed in the gynecologic/urologic communities to be caused by a decreased ‘testosterone effect’, such treatment may be beneficial. This decreased ‘testosterone effect’ can be due to normal aging, contraceptive medications, anti-depressant medication, a lowered cholesterol diet or a decreased percentage of body fat as a result of a rigorous exercise regime. This present invention defines those three above-stated specific nutritional supplements with specific physiological effects well documented in the peer reviewed medical literature. This combination of nutritional supplements increases the testosterone effect in both the brain as well as in the clitoral tissues, to improve a woman's libido, without prescription testosterone preparations, or the uncertainties of non-medically proven herbal remedies.

### BACKGROUND OF THE INVENTION

1. Field of the Invention

This application relates to dietary supplements for women to improve her libido, and more particularly to the use of an oral, daily-dosed nutritional supplement of DHEA (Dihydroepiandosterone), Yohimbe, and L-Arginine, to increase that woman's libido and sexual responsiveness, and is a continuation-in-part application of my earlier co-pending U.S. patent applications, Ser. No. 09/736,973, filed Dec. 14, 2000, entitled “Clitoral Sensitizing Arrangement” and Ser. No. 09/968,055 filed Oct. 1, 2001, entitled “Treatment of Interstitial Cystitus Using Topical Application of Menthol and L-arginine” both of which are incorporated herein by reference in their entirety.

2. Prior Art

In the 1993 Journal of Sexual Marital Therapy Vol. 19 NO.1, Dr. Helen Singer Kaplan reported the direct association between a woman's testosterone level and that woman's libido in “The Female Androgen Deficiency syndrome”, Dr. Kaplan reported,

“The loss of testosterone within androgen-deficient female patients not only caused the lack of libido, but also profoundly decreased the ability to have an orgasm even with excessive clitoral stimulation. Orgasms in these patients, even when achieved were described as genitally localized and only of minor intensity. Both problems, libido and more importantly the ability to become aroused and achieve a satisfactory orgasm, were resolved by treatment with testosterone injections. Testosterone is essential for adequate libido and for the ability to achieve meaningful orgasms.”

The more recent reports Dr. Susan Davies writes,

“Testosterone is an important component of female sexuality, enhancing interest in initiating sexual activity and response to sexual stimulation.”

in the March 2001 Journal of Reproductive Medicine article “Testosterone Deficiency in Women”. It is well established in the urologic and gynecologic communities that testosterone is essential for a woman's sexuality as reported in over one hundred peer-reviewed medical articles. In both of these quotes it is important to understand the reference of ‘libido’ as a desire or brain-responsible emotion and ‘sexual responsiveness’ as a clitoral or orgasm related response. Brain and Clitoris, anatomically separate but neurally and hormonally interactive!

Testosterone significance in women: A woman has one third to one sixth of the serum level of Testosterone as a same aged man in normal situations. A woman's testosterone is produced 25% by her ovaries, and 75% by the adrenal glands and the peripheral fat conversion of DHEA (Dihydroepiandosterone) into androgens. Dr. Kaplan reports that a woman's testosterone level declines ‘normally’ 70% from age 30 to age 60. The medical literature also associates the decline of libido with contraceptive medications, due to the increase in sex-binding globulins in the blood that transports testosterone, anti-depressant medications as well as with normal aging.

DHEA—Dihydroepiandosterone significance in women: DHEA is not only the precursor of the sex hormones, testosterone and estrogen, it is also a nutritional supplement available for oral consumption. In the Journal of Society for Gynecological Investigation Vol. 8 from March 2001, Dr. Cristen Slater reports,

“Potential use of DHEA supplementation for Estrogen and Androgen Replacement in post-menopausal women” “After six months of oral DHEA supplementation (25 mg/day) post-menopausal women's estrogen and testosterone levels returned to premenopausal levels”.

Menopause is when a woman stops having menstrual periods, signifying the cessation of ovarian functioning. Post menopausal levels of estrogen are negligible and most menopausal levels of androgens are normally low. Peri-menopause is defined as the years just prior to and after a woman initiates menopause. Normal age range for menopause are reported as age 42 to age 54 with the average at age 49. “Peri-menopausal transition may last as long as seven years,” reports Dr. James Simon in “Safety of Estrogen/Androgen Regimes”, also in the March 2001 Journal of reproductive Medicine. Therefore, from age 35 to 60 women can normally experience the effects of decreased libido from decreased testosterone. These effects of decreased libido from decreased testosterone can occur abruptly at any age, if a woman's ovaries are surgically removed. DHEA can return these decreased testosterone levels, as well as estrogen, to pre-menopausal levels.

Yohimbe's significance: DHEA offers a solution to increase the production of testosterone, but in the end, testosterone's effect upon tissues, especially brain and clitoral tissues, depends upon the delivery of the testosterone to these tissues. By definition, a hormone is a protein that is produced in an endocrine gland, circulates in the blood stream, and has its effect at a distant target tissue. The testosterone hormone is transported in the blood by the sex-binding globulin that is produced in the liver, and in a free non-bound state. A normal woman not on birth control pills normally has 1% free unbound testosterone. It is the free unbound testosterone that has an effect in target tissues. The normally 99% bound testosterone is ineffective at target tissues. Yohimbe is a nutritional supplement. As an indolic alkaloid found in the bark of the corgunanthe yohimbe plant, yohimbe has a history of popular use for aphrodisiac properties. L. Paul Sondra reported of “The Role of Yohimbe for the Treatment of Erectile Impotence” in the 1990 Journal of Sex and Marital Therapy that 38% of men reported subjective improvement over impotence while treated with 5 mg. of yohimbe in a double blind study. Yohimbe selectively binds to the sex-binding globulin to increase the free circulatory testosterone. This effect is quite dramatic, for women with a 1.0% of free serum testosterone report a normal libido, women on birth control pills with a 0.3% free serum testosterone report an absent libido, and women with a 2.0% free serum testosterone not only report a strong libido, but they will experience virilizing effects like growing sideburns and a mustache. Virilizing effects are, anatomical evidence of increased testosterone effect such as acne, male pattern hair growth, increased muscle mass, lower tonal voice, and clitoralmegaly (increased clitoral size) in women. Clitoralmegaly is evidence of either an inborn error of metabolism like an 21-hydrolase deficiency, or steroid use as in body building women. The opposite of virilizing effects can be seen in treatments for increased hair growth, hirsuitism, and acne, such as contraceptive medications that increase sex binding globulins and decrease free serum testosterone. Certain contraceptives are even FDA approved to treat acne, namely Ortho-TriCyclin. Again, both the total testosterone (free and bound) circulating in the serum and the percent of that total testosterone that is ‘free’ will determine the tissue effect of testosterone. DHEA can increase the total testosterone, free and bound, and the Yohimbe can increase the specific free compound, to effectively increase libido and sexual responsiveness in the brain and the clitoral tissues respectively. To again quote Dr. Kaplan,

“Both problems, libido and more importantly the ability to become aroused and achieve a satisfactory orgasm, were resolved (in androgen deficient females) by treatments with testosterone injections.”

L-arginine significance: L-arginine is a semi-essential amino acid that is also a nutritional supplement. L-arginine is obtained by a healthy diet rich in vegetables. The L-arginine is stored in tissues in minute quantities. L-arginine is the substrate for the nitric-oxide synthase pathway where nitric oxide is produced. The nitric oxide not only causes vasodilatation but is a local cellular signaler that can stimulate vasodilatation and increased cellular activity to promote tissue growth and increased tissue sensitivity. This increased tissue sensitivity is related to the increased number or sensitivity of specific receptors, or recognition sites, for hormones such as testosterone. A target tissue for a specific hormone is defined by the presence of receptors to that hormone within the target tissue. My U.S. patent applications, Ser. No. 09/736,973, filed Dec. 14, 2000, entitled “Clitoral Sensitizing Arrangement” and Ser. No. 09/968,055 filed Oct. 1, 2001, entitled “Treatment of Interstitial Cystitus Using Topical Application of Menthol and L-arginine” teache about the three isoforms of the nitric oxide synthase enzyme and relates how the inducible isoform can be induced to increase cellular activity by increasing the substrate, 1-arginine. R. A. Smulders et al, in Homodynamic and biochemical responses to L-arginine and L-lysine infusions in normal subjects: L-arginine-induced vasodilatation cannot be explained by non-specific effects of cationic amino acids (Clinical Science, 1997, 92) in the conclusion, state:

“Therefore, our results indirectly support the hypothesis that an increased availability of L-arginine, the substrate for the NO synthase, can increase basal NO synthesis” (NO is Nitric Oxide)

This increased L-arginine substrate can have an effect in the brain (thalamus) and in the clitoral tissues. Shan and Salt in the 1997 European Journal of Neuroscience 9, in an article entitled, Modulation of Sensory and excitatory amino acid responses by Nitric Oxide donors and glutathione in the ventrobasal thalamus of the rat reports:

“In the ventrobasal complex of the thalamus (brain), the precursor of Nitric Oxide synthesis, L-arginine causes enhancement of excitatory amino acid responses and sonato sensory transmission.”

In the clitoris, the L-arginine substrate activation is the subject of the article by Burnett A C et al, Immuno histo chemical description of nitric oxide synthase isoforms in human clitoris. Journal of Urology 158: 75-78, 1997.

The clinical aspects of L-arginine supplementation to increase both libido (brain) and clitoral responsiveness can be somewhat implied by the 2001 report on ARGINMAX™, a nutritional supplement that contains L-arginine, but also includes several herbal remedies. Ito, Ty et al reported both an increased libido and sexual responsiveness in, a double-blind placebo controlled study of ARGINMAX™, a nutritional supplement for enhancement of female sexual functions, Journal of Sex and Marital Therapy 2001; 27: 541-579. The described enhancement of a woman's sexual function again reports, both the increase in libido (brain) and sexual responsiveness (the ability to achieve orgasm) directly related to the clitoral functions. ARGINMAX™ does not contain DHEA, testosterone or any of its precursors.

### PREFERRED EMBODIMENTS OF THE INVENTION

The present invention thus comprises a preferably orally administered compound of a novel formulation by combining three specific nutritional supplements with specific physiological effects well documented in the peer reviewed medical literature. This combination of nutritional supplements increases the testosterone effect in both the brain as well as in the clitoral tissues, to improve a woman's libido, without prescription testosterone preparations, or the uncertainties of non-medically proven herbal remedies.

The formulation of that compound is as follows:

| | Range of | | Preferred |
|---|---|---|---|
| Preferred ingredients | Dosage/Day | | Dosage/Day |
| DHEA | 5-50 | mg | 25 mg |
| Yohimbe | 1-10 | mg | 5 mg |
| L-arginine | 100-2000 | mg | 500 mg |

Such a compound is intended to be provided as an orally received formulation in a carrier base of for example, soybean oil, lecithin and wax mixture, taken orally for example, once or twice a day. Other examples comprise DHEA-S (Dihydroepiandesterione-Sulfate) also available as a nutritional supplement.

The present invention thus comprises a nutritional supplement for increasing a mammalian female's libido, comprising a compound of DHEA, Yohimbe and L-arginine. The compound is preferably ingested daily. The invention also includes a method to increase both the libido and sexual responsiveness in a mammalian female, comprising the steps of: administering a compound having a combination of DHEA, Yohimbe and L-arginine to that female. The DHEA may be administered to the female in a dosage range of for example, about 5-50 mg./day. The Yohimbe may be administered to the female in a dosage range of for example, about 1-10 mg./day. The L-arginine may be administered to the female in a dosage range of for example, preferably about 100-2000 mg./day. The DHEA may be administered to the female in a preferred dosage of for example, about 25 mg./day. The Yohimbe may be administered to the female in a dosage of for example, about 5 mg./day. The L-arginine may be administered to the female in a dosage of for example, about 500 mg./day. The compound may be administrated to the female orally. The compound may be administrated for example sublingually.

The invention also includes a method of treating decreased libido and sexual responsiveness in a mammalian female by: administering a compound having a combination of ingredients which are comprised of DHEA, Yohimbe and L-arginine, to the female. The decreased libido may be effected by contraceptive administration in the female. The decreased libido may be effected by administration of an anti-depressant drugs in the female. The decreased libido may be effected by decreased body fat and decreased peripheral conversion of testosterone precursors into testosterone in the female. The decreased libido may be effected by decreased production of testosterone precursors because of a low cholesterol diet in the female.

The invention also includes a method to increase total serum testosterone and unbound serum testosterone in a mammalian female to increase libido and sexual responsiveness in that female by the step of: administering a compound of DHEA and Yohimbe to the female. The compound may include L-arginine. The compound may contain DHEA in a range of about 5-50 mg., Yohimbe in a range of about 1-10 mg., and L-arginine in a range of about 500-2000 mg.

The invention also includes methods of treating age-related decreased libido and sexual responsiveness in a mammalian female, the method comprising: administering a compound having a combination of ingredients which are comprised of DHEA, Yohimbe and L-arginine, to the female; methods of treating surgical castration related decreased libido and sexual responsiveness in a mammalian female, the method comprising: administering a compound having a combination of ingredients which are comprised of DHEA, Yohimbe and L-arginine, to the female; methods of treating decreased body fat relative to the consequences of decreased peripheral conversion of testosterone precursors into testosterone, in a mammalian female, comprising: administering a compound having a combination of ingredients which are comprised of DHEA, Yohimbe and L-arginine, to the female; methods of treating the decreased production of testosterone precursors due to a low cholesterol diet in a mammalian female, comprising: administering a compound having a combination of ingredients which are comprised of DHEA, Yohimbe and L-arginine to the female; methods of increasing the nitric oxide synthase enzyme activity to increase libido and sexual responsiveness in a mammalian female, comprising: administering to the female, a nutritional supplement to increase both total serum testosterone and unbound serum testosterone in the female, wherein the nutritional supplement comprises a compound having a combination of ingredients which are comprised of DHEA, Yohimbe and L-arginine.

I claim:

1. A nutritional supplement for increasing a mammalian female's libido, comprising a compound of DHEA, Yohimbe and L-arginine.

2. The nutritional supplement as recited in claim 1, wherein said compound is ingested daily.

3. A method to increase both the libido and sexual responsiveness in a mammalian female, comprising the steps of:
administering a compound having a combination of DHEA, Yohimbe and L-arginine to said female.

4. The method as recited in claim 3, wherein said DHEA is administered to said female in a dosage range of 5-50 mg./day.

5. The method as recited in claim 3, wherein said Yohimbe is administered to said female in a dosage range of 1-10 mg./day.

6. The method as recited in claim 3, wherein said L-arginine is administered to said female in a dosage range of 100-2000 mg./day.

7. The method as recited in claim 3, wherein said DHEA is administered to said female in a preferred dosage of about 25 mg./day.

8. The method as recited in claim 3, wherein said Yohimbe is administered to said female in a dosage of about 5 mg./day.

9. The method as recited in claim 3, wherein said L-arginine is administered to said female in a dosage of about 500 mg./day.

10. The method as recited in claim 3, wherein said compound is administrated to said female orally.

11. The method as recited in claim 3, wherein said compound is administrated to said female by sublingual absorption.

12. A method of treating decreased libido and sexual responsiveness in a mammalian female by:
administering a compound having a combination of ingredients which are comprised of DHEA, Yohimbe and L-arginine, to said female.

13. The method as recited in claim 12, wherein said decreased libido is effected by contraceptive administration in said female.

14. The method as recited in claim 12, wherein said decreased libido is effected by administration of an anti-depressant in said female.

15. The method as recited in claim 12, wherein said decreased libido is effected by decreased body fat and decreased peripheral conversion of testosterone precursors into testosterone in said female.

16. The method as recited in claim 12, wherein said decreased libido is effected by decreased production of testosterone precursors because of a low cholesterol diet in said female.

17. A method to increase total serum testosterone and unbound serum testosterone in a mammalian female to increase libido and sexual responsiveness in said female by the step of:
administering a compound of DHEA and Yohimbe to said female.

18. The method as recited in claim 17, wherein said compound includes L-arginine.

19. The method as recited in claim 18, wherein said compound contains said DHEA in a range of about 5-50 mg., said Yohimbe in a range of about 1-10 mg., and said L-arginine in a range of about 500-2000 mg.

20. A method of treating age-related decreased libido and sexual responsiveness in a mammalian female, said method comprising:
administering a compound having a combination of ingredients which are comprised of DHEA, Yohimbe and L-arginine, to said female.

21. A method of treating surgical castration related decreased libido and sexual responsiveness in a mammalian female, said method comprising:
administering a compound having a combination of ingredients which are comprised of DHEA, Yohimbe and L-arginine, to said female.

22. A method of treating decreased body fat relative to the consequences of decreased peripheral conversion of testosterone precursors into testosterone, in a mammalian female, said method comprising:
administering a compound having a combination of ingredients which are comprised of DHEA, Yohimbe and L-arginine, to said female.

23. A method of treating the decreased production of testosterone precursors due to a low cholesterol diet in a mammalian female, said method comprising:
administering a compound having a combination of ingredients which are comprised of DHEA, Yohimbe and L-arginine, to said female.

24. A method of increasing the nitric oxide synthase enzyme activity to increase libido and sexual responsiveness in a mammalian female, said method comprising:
administering to said female, a nutritional supplement to increase both total serum testosterone and unbound serum testosterone in said female, wherein said nutritional supplement comprises a compound having a combination of ingredients which are comprised of DHEA, Yohimbe and L-arginine.
